(19) United States
(12) Patent Application Publication
(10) Pub. No.: US 2015/0133844 A1
(43) Pub. Date: May 14, 2015

(54) EASY REMOVAL ADHESIVE ARTICLE

(71) Applicant: Johnson & Johnson Consumer Companies, Inc., Skillman, NJ (US)

(72) Inventor: Shannon Montulet, Kendall Park, NJ (US)

(73) Assignee: Johnson & Johnson Consumer Companies, Inc., Skillman, NJ (US)

(21) Appl. No.: 14/075,353

(22) Filed: Nov. 8, 2013

(51) Int. Cl. A61F 13/02; A61F 13/00

(52) U.S. Cl. CPC A61F 13/0259; A61F 13/0206; A61F 13/00063
USPC 602/44; 602/57; 602/46; 602/45; 602/48

(57) ABSTRACT

An elongate adhesive article having a length substantially greater than a width includes a backing layer having an adhesive layer and a single weakened region formed of a removable strip of backing layer that is oriented substantially perpendicular to the article length. The removable strip of backing layer is at least partially defined by a pair of lines of weakness separated by a gap of between about 2 and about 15 mm. The elongate article may include an absorbent pad in the form of an adhesive bandage or sticking plaster. A method of using such an elongate article includes the steps of applying the article to the skin of a mammal, removing a predefined strip from the article; and removing the remainder of the article from the skin.

### FIELD OF THE INVENTION

The present invention relates to adhesive articles that are easily removable from the substrate on which they are disposed. More particularly, the invention relates to adhesive articles such as medical tapes and wound covers with easy to tear regions for removal from the skin of a mammal.

### BACKGROUND OF THE INVENTION

Adhesive articles such as tapes and wound covering bandages are well known in the art and are used for various medical applications for humans and other mammals and for sports protection of humans. In the case of wound covering bandages, a sterile wound-covering pad contacts the wound, and a backing layer coated with a pressure sensitive adhesive provides secure attachment of the bandage to bare skin adjacent to a wound.

Recently, adhesives have been developed for adhesive articles such as bandages that do not peel off even when the bandage is wetted by sweat or swimming activity. The corners of the article are often shaped to prevent easy release of the bandage.

All these developments make removal of the bandage difficult, especially when the area of the skin is covered with hair. Bandage removal is typically so painful that most users remove the bandage by pulling sharply, as fast as possible, to minimize the duration of pain. This action sometimes causes further cuts and wounds to the user, especially when the bandages are repeatedly removed and replaced on the wound site.

When bandages are used to treat small appendages (fingers or toes), the bandage is often wrapped around the appendage, and the backing layer overlaps. In these cases, the adhesion of the overlap is stronger than the adhesion of the bandage to the skin. Frequently, to avoid pulling the bandage sharply, a user employs a sharp instrument such as a knife, scissor, or even a scalpel to assist in removing the bandage from the skin. This procedure could cut the user seriously enough to warrant stitches.

To overcome the issue of difficult removal of adhesive articles, others have used “pull-tabs”, pieces of backing with no adhesive jutting out from edges of the backing. In other cases, the backing has regions of low adhesion to help in removal from the skin. Another easy-removal method is to have solid beads containing oil that can be broken to release the oil into the adhesive region to disrupt the adhesion.

For example, Silber, U.S. Pat. No. 4,526,166, purports to disclose a bandage including first and second carrier strips. The first strip includes a liquid absorbent, soft, pad of material which is to be placed over a wound. The second strip carries an adhesive to be placed onto the patient's skin at a location spaced from the wound. The two strips have ends which are connected by a releasable attachment forming a third element such as a tear string which is easily manipulable to free the strips from one another. This enables lifting of the pad and first strip independently of the second strip, so that force applied to the second strip to free it from the skin is not transferable to the first strip and wound.

Stebbings et al., U.S. Pat. No. 7,078,582, purports to disclose pressure sensitive adhesive articles and methods, particularly stretch removable adhesive articles that are preferably for use in adhering to skin or like delicate surfaces. Preferably, stretch removability of the article occurs as a result of the selection of a stretch removable pressure sensitive adhesive. In one embodiment of the articles and methods, the adhesive and backing delaminate upon removal. In another embodiment of the articles and methods, the backing includes a predefined tab located in a central location of the backing.

These solutions have failed to completely solve the issue of difficult removal of adhesive articles. Pull tabs can accidently catch and release the article prematurely. The beads with oil can rupture accidently and release the article prematurely. And controlling the amount of adhesive in various regions of the backing is difficult.

There remains a need in the art for adhesive articles that adhere well to mammalian skin and are easily removable therefrom at will without causing excessive pain or risk of further damage. This need has been exacerbated by the inherent difficulty in creating a bandage adhesive that bonds well and, at the same time, can be readily removed with minimal difficulty during peeling of the bandage away from mammalian skin.

### SUMMARY OF THE INVENTION

Surprisingly, we have found a novel way to improve the ease with which an adhesive article may be removed from a user's skin. In one aspect of the invention, an elongate adhesive article having a length substantially greater than a width, includes a backing layer having an adhesive layer and a single weakened region formed of a removable strip of backing layer that is oriented substantially perpendicular to the article length. The removable strip of backing layer is at least partially defined by a pair of lines of weakness separated by a gap of between about 2 and about 15 mm. The elongate article may include an absorbent pad in the form of an adhesive bandage or sticking plaster.

In another aspect of the invention, a method of using such an elongate article includes the steps of applying the article to the skin of a mammal, removing a strip from the article; and removing the remainder of the article from the skin. The step of removing the strip includes gripping the removable strip of backing layer proximate the first side edge of the article and pulling the removable strip of backing layer away from the skin to which it is applied, thereby fracturing the backing layer along the lines of weakness. The removable strip is then removed from the adhesive article, leaving a remainder of the adhesive article adhered to the skin.

### BRIEF DESCRIPTION OF THE DRAWINGS

An embodiment of this invention will now be described in greater detail, by way of illustration only, with reference to the accompanying drawings, in which

FIG. 1 is a top plan view of an elongate adhesive article of the present invention.

FIG. 1a is an enlarged portion of the elongate adhesive article of FIG. 1.

FIG. 2 is a side view of the article of FIG. 1 taken along the x---x plane.

FIG. 3 is a top plan view of a second embodiment of an elongate adhesive article of the present invention.

FIG. 3a is an enlarged portion of the elongate adhesive article of FIG. 3.

FIG. 4 is a side view of the article of FIG. 3 taken along the x---x plane.

FIG. 5 is a top view of an elongate adhesive article in the form of a wound cover of the present invention.

FIG. 6 is a cross-sectional view of the wound cover of FIG. 5 taken along the x---x plane.

FIG. 7 is an end view of the wound cover of FIG. 5 prior to lifting of the easy to tear region.

FIG. 8 is an end view of the wound cover of FIG. 5 after the easy to tear region has been lifted.

FIG. 9 is a top view of a wound cover according to the invention after the easy to tear region has been partially removed.

FIGS. 10a-c are a representation of one removal method of the elongate adhesive article of the present invention from a user's finger. FIG. 10a shows the second embodiment elongate adhesive article on a user's finger. FIG. 10b shows the elongate adhesive article after the easy to tear region has been lifted. FIG. 10c shows the elongate adhesive article partially removed from a user's finger.

### DETAILED DESCRIPTION OF THE INVENTION

Again, adhesive articles that adhere well to for human and other mammalian skin are often difficult to remove, especially when they are applied around fingers and toes. Therefore, we have provided novel elongate adhesive articles that are easily removable from mammalian skin without causing excessive pain or risk of further damage.

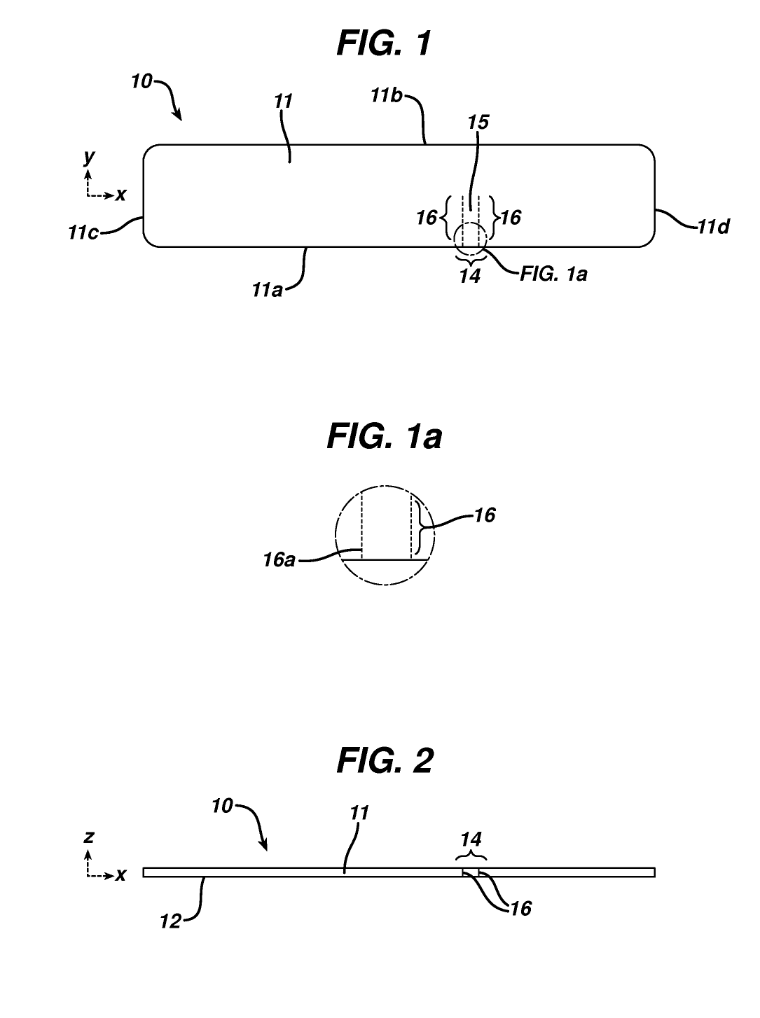

FIGS. 1 and 2 illustrate a first embodiment of an elongate adhesive article of the present invention. The elongate adhesive article 10 comprises backing layer 11 having a length substantially greater than a width and with a first and second surface, and an adhesive layer 12 associated with the first surface. Backing layer 11 has first side edge 11a, second side edge 11b, first end 11c, and second end 11d. Article 10 has a single weakened region 14 comprising a removable strip 15 of backing layer that is oriented substantially perpendicular to the article length. The removable strip 15 is at least partially defined by a pair of weakness 16 which originate from first side edge 11a, and are generally disposed across the width of the elongate article. To aid in the removal of the elongate adhesive article 10 from a surface to which it is adhered, a user would fracture the lines of weakness 16 starting from first side edge 11a, along their length (across the width of the elongate element, the “y-axis”), and remove the strip 15 in the region between the lines of weakness 16. The user can then use the torn edges remaining on adhesive article 10 as a tab to aid in the removal of the remaining pieces of article 10.

Backing layer 11 may have various shapes, including but not limited to, rectangular, oval, ovoid, or oblong. The shape of the elongate adhesive article 10 is defined by the shape of backing layer 11. Backing layer 11 may be thin, highly flexible or deformable, water-impervious, and clear or opaque. In general, the thickness of backing layer 11 is between about 0.05 to 0.2 millimeter (“mm”) to achieve the forming and flexing characteristics desired.

It is desired for the material used in backing layer 11 to be both conformable to the contours of the body, and flexible so as to permit free movement of the body part wearing the product. Further, backing layer 11 could be lightweight, and may be elastic (elastomeric) in character. It can be a woven or nonwoven fabric, a film or a foam. Polymeric materials useful in forming the backing layer 11 include polyolefin (such as polyethylene), polyurethane, and polyvinylchloride. Other examples of backings include, but are not limited to, nonwoven, woven, or knitted fabrics such as cotton, polyester, polyurethane, rayon and the like.

A polyethylene film may be used as backing layer 11, and particularly effective results can be achieved with stretchable, elastomeric films formed of polyurethane, which has the further advantage of gas (including water vapor) transmissibility. It is to be understood, however, that other flexible, water insoluble polymeric films known in the art may be used. Furthermore, backing layer 11 may be formed from closed-cell polymeric foam, particularly one with an integral skin covering the side facing away from the skin of the user. Foam layers formed of polyurethane or polyethylenes are suitable, while other polymeric foams having similar properties may be used. In addition, backing layer 11 may be made from other polyolefins, vinyl polyethylene acetate, textile non-woven fabrics, rubber, or other materials known in the adhesive article art. Polymers used to make backing layer 11 used in bandages of the present invention may exhibit viscosity of about 500 to 500,000 centipoises at temperatures of about 190° C., or about 1,000 to 30,000 centipoises at temperatures of about 190° C., or about 3,000 to 15,000 centipoises at temperatures of about 190° C. Backing layer 11 may be impermeable to liquid, but permeable to gas, which allows the wound and the skin to which the elongate adhesive article 10 of the present invention is adhered to breathe. In one embodiment, backing layer 11 may have pores of such a size that will allow only the passage of gases, which have molecules of extremely small size. Finally, one can conceive of a backing layer that is perforated for more ventilation of the skin. Perforations may be circular in area and have a range of diameters, such as from about 0.1 to about 0.8 millimeters. However, backing layer 11 may be totally impermeable to gases, when necessary.

In one preferred embodiment, the backing layer comprises a woven fabric having either a warp or weft oriented substantially across the width of the elongate article. Thus a tear propagated across the elongate article will tend to continue until the article is separated by removal of the removable strip 15. In another embodiment, the backing layer comprises an oriented polymer in which a tear propagated across the elongate article will tend to continue until the article is again separated by removal of the removable strip 15.

Again, the length of the backing layer is substantially greater than the width. Preferably, the width of backing layer 11 is at least about 10 mm, more preferably at least about 15 mm or at least about 20 mm. In a preferred embodiment, the width of backing layer 11 is less than about 100 mm, more preferably, less than about 70 mm, and most preferably, less than about 40 mm. Thus, the width of backing layer 11 may be between about 10 mm and 100 mm, preferably between about 15 mm and 70 mm, more preferably between about 20 mm and 40 mm. In one embodiment the width of backing layer 11 is about 25 mm.

Article 10 has a single weakened region 14 at least partially defined by two lines of weakness 16 which originate from first side edge 11a, and are disposed along the y-axis. The resulting removable strip has a width (defined by the maximum distance between the two lines of weakness 16) between about 2 mm and 15 mm, preferably between about 2 mm and 10 mm, more preferably between about 2 mm and 6 mm, and most preferably about 4 mm.

While FIG. 2 shows the line of weaknesses 16 are formed by weakness elements 16a that pass fully through backing layer 11 and adhesive layer 12 along the z-axis. It is to be understood that in other embodiments, the lines of weakness 16 may be defined by scoring, a thinning of the backing layer from its original thickness. In other embodiments, at least one of the two lines of weakness 16 may be scored, while the other line of weakness 16 includes weakness elements 16a that are fully perforated.

Preferably, the lines of weakness 16 are substantially parallel. However, it may be acceptable for the lines to converge or diverge as long as they provide a removable strip 15 of backing layer 11.

Weakness elements 16a may be any shape, including circular, elliptical, linear, arrow- or V-shaped, Y-shaped, S-shaped, chevron, a partial-chevron, and a dot pattern. In some embodiments, a simple linear pattern of linear cuts is used, in which the cuts are aligned to create a single, broken separation line that extends laterally across the elongate adhesive article 10.

In some embodiments, the weakness elements are of equal length. This is shown in the enlarged view if FIG. 1a. The length of the weakness elements may range from about 0.1 mm to about 5 mm, alternatively from about 0.25 mm to about 3 mm, or even from about 0.5 mm to about 1 mm. The gap between weakness elements may be uniform or variable. The gap between the weakness elements may range from about 0.1 mm to about 5 mm, preferably, from about 0.25 mm to about 3 mm, and most preferably, between about 0.5 mm and about 1 mm.

The sizes of weakness elements 16a and the distance between them help to determine the force required to tear and remove the removable strip 15.

As shown in FIG. 1, the lines of weakness 16 originate from first side edge 11a, are generally disposed along the y-axis, and terminate at point between first side edge 11a and second side edge 11b. As shown in the figure, the lines of weakness 16 span approximately half (50%) of the width of the backing layer. It is to be understood that in various embodiments, lines of weakness 16 may span substantially all of the width of the backing layer, or some amount less than the width of the backing layer. In one preferred embodiment, the lines of weakness span about 75% of the width. In another embodiment, the lines of weakness span about 33% or even 25% of the width of the backing layer.

FIG. 1 also shows lines of weakness 16 originating at first side edge 11a. It is to be understood that in other embodiments, lines of weakness 16 may originate at a point part way between first side edge 11a and second side edge 11b. For example, the lines of weakness may be spaced in slightly, such as between about 1% and about 5% of the backing layer width, from the side edges to prevent undesired premature removal of the removable strip. In another embodiment, the lines of weakness may be spaced at a greater distance from the side edge; perhaps as much as 10% to 20% of the backing layer width.

Again, the backing layer 11 of the elongate adhesive article 10 has an adhesive layer associated with, preferably disposed on, the first surface thereof. In general, any of a variety of pressure-sensitive adhesives can be utilized as adhesive layer 12. In particular, pressure-sensitive adhesives that are biocompatible with human skin are typically utilized. In some embodiments, an adhesive of the present invention may also be either generally water soluble or generally insoluble, or dispersible in an aqueous environment. For instance, commercially available dispersible pressure-sensitive adhesive is sold under the trade name of HL-9415-X and is available from H.B. Fuller Company. Another suitable adhesive includes about 10-75% by weight of a polyalkyloxazoline polymer, 10-75% by weight of a functional diluent comprising a hydroxy compound or a carboxylic acid compound, and 5-50% by weight of a tackifier.

Adhesive layer 12 may comprise hydrocolloids. The hydrocolloid element used may be any substance that has a good performance in this utilization, as for example, sodium carboxymethylcellulose, pectin, xanthan gum, polysaccharides, sodium or calcium alginates, chitosan, seaweed extract (cageenan), polyaspartic acid, polyglutamic acid, hyaluronic acid or salts and derivatives thereof, among others.

Hydrocolloids, just as sodium carboxymethylcellulose and pectin, among others, are agents that form gels as soon as they come into contact with the bodily fluids from the wound. When used in adhesive bandages, these hydrocolloids are combined with elastomers and/or adhesives. Preferably, the adhesive bandage should provide a humid environment but without saturation, cicatrisation, which is a situation suitable for acceleration of the healing,

Adhesive layer 12 may be any conventional adhesive known for such use, as for example pressure acrylic adhesives, among others. Additionally, such an adhesive may contain a resin for increasing adhesion, a cohesion increasing agent, an absorption agent (preferably a polyacrylate superabsorbent, a polyacrylate salt superabsorbent or a mixture thereof), a plasticizer and optionally a pigment. The adhesive layer may further be configured in discontinuous patterns, arranged in lines, screen, spray or any other which a person skilled in the art understands as discontinuous, composed by an elastomeric base.

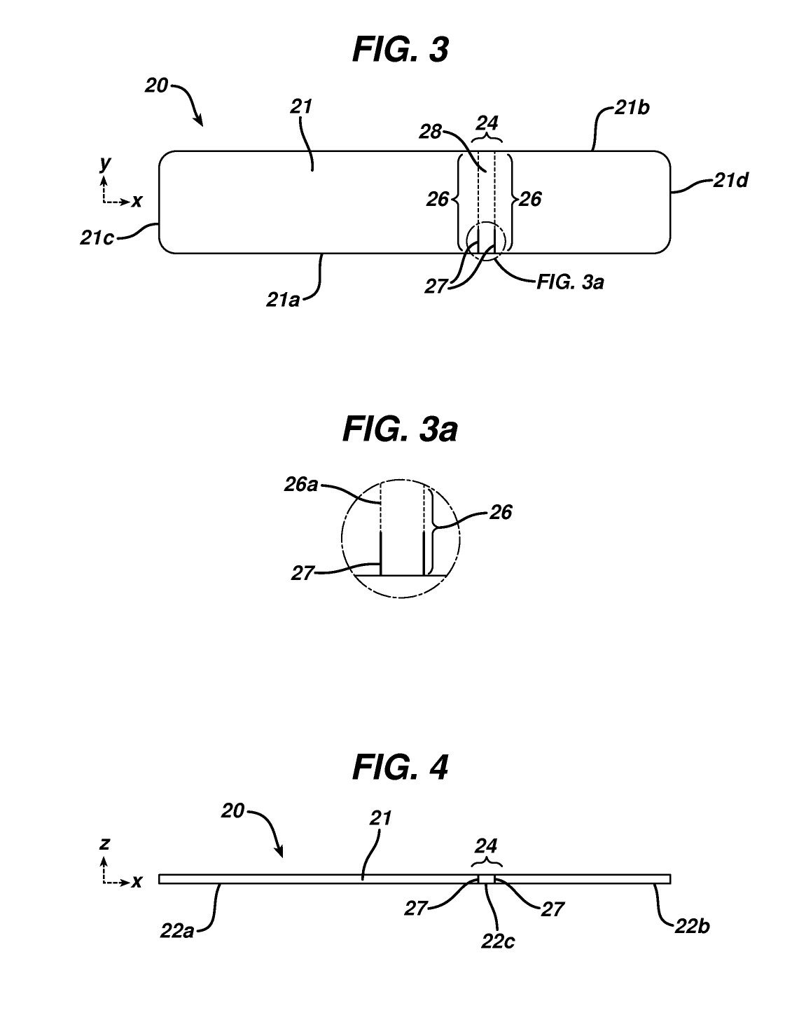

FIGS. 3 and 4 illustrate a second embodiment of an elongate adhesive article of the present invention. The elongate adhesive article 20 comprises backing layer 21 with a first and second surface, and an adhesive layer 22 associated with the first surface. Backing layer 21 has first side edge 21a, second side edge 21b, first end 21c, and second end 21d. Article 20 has single weakened region 24 comprising a removable strip of backing layer that is at least partially defined by a pair of lines of weakness 26 and a pair of slits 27 which originate from first side edge 21a, and are disposed along the y-axis. Weakness elements 26 and slits 27 result in a removable strip 28 across the elongate adhesive article 20.

To aid in the removal of the elongate adhesive article 20 from a substrate on which it is disposed, user would lift an edge of the removable strip 28 from the substrate starting between the pair of slits 27 using for example, a finger nail. In this manner, the pair of slits 27 acts as a “pick point”, or “starting point”. User would then tear lines of weakness 26 starting from first side edge 21a, along their length across the backing layer, and remove the removable strip 28. The user can then use the torn edges remaining on the elongate adhesive article 20 as a tab to aid in the removal of the remaining pieces of article 20 from the substrate.

Details of backing layer 21, adhesive layer 22, and lines of weakness 26, and slits 27 are the same as those discussed in the first embodiment (FIGS. 1 and 2). In this embodiment, adhesive layer 22, disposed on the first surface of the backing layer is shown as having two portions: a first portion 22a having a first peel strength and corresponding to the removable strip 28, and a second portion including first adhesive section 22b and second adhesive section 22c having a second peel strength. The peel strength may be determined using PTSC-101 (Test Method F, 90° angle on a standard steel plate, Issued October 2000, Revised October 2003 & May 2007). In some embodiments, the peel strength of the two portions is substantially identical. In other embodiments, the peel strength of the second portion (including the first and second adhesive sections 22b and 22c) is greater than that of the first portion 22a. In some embodiments, the peel strength of the first portion 22a is about 75% of the peel strength of the second portion. In other embodiments, the peel strength of the first portion 22a is about 50% of the peel strength of the second portion, and more preferably, less than about 25% of the second portion. In yet another embodiment, there is substantially no adhesive on the first portion of the first surface the backing layer. The different degrees of adhesion between the portions of the first surface of the backing layer can be accomplished by using a second adhesive with lesser adhesion on the first portion 22a. Alternatively, the adhesive layer may be configured in discontinuous patterns on backing layer 21, with less, or no adhesive applied on the first portion 22a.

As shown in FIG. 3, lines of weakness 26 terminate proximate second side edge 21b. It is to be understood that, as in the embodiments relating to FIGS. 1 and 2, above, lines of weakness 26 may span all or part of the width of the backing layer.

As shown in FIG. 3 and the detailed view of FIG. 3a, the slits 27 that define the “pick point” of the removable strip 28 originate at first side edge 21a of backing layer 21. It is to be understood that in other embodiments, slits 27 may also originate at second side edge 21b of backing layer 21. In still other embodiments, two sets of slits 27 may originate at both first side edge 21a and second side edge 21b of backing layer 21.

Slits 27 may be any shape, including circular, elliptical, linear, arrow- or V-shaped, Y-shaped, S-shaped, chevron, or partial-chevron. In some embodiments, a simple linear pattern of slits is used, in which the slits are aligned to create a single, broken separation line that extends laterally across the elongate adhesive article 10.

Slits 27 may be perpendicular to the side edge 11a of the backing material or they may be arranged at an angle other than 90°. However, the lines of weakness 26 associated with such angled slits are preferably substantially perpendicular.

In some embodiments, slits 27 are of equal length. The length of the slits may range from about 0.1 mm to about 5 mm, preferably from about 0.25 mm to about 3 mm. If it is desired to minimize the length of the slit and to reduce the likelihood of premature removal of the removal strip, one may desire to limit the slit to a length of between about 0.5 mm and about 1 mm.

Also, as shown in FIG. 3, the lines of weakness comprises weakness elements 26a that pass fully through backing layer 21 and adhesive layer 22 along the z-axis. It is to be understood that in other embodiments, the lines of weakness 26 may be scored, either continuously or in a broken line pattern.

As mentioned above, to aid in the removal of the elongate adhesive article 20 from a substrate to which it is adhered, user lifts backing layer 21 starting between the pair of slits 27. User then tears perforations 26, starting from first side edge 21a, along their length (along the y-axis), and removes the removable strip 28. In embodiments where the peel strength of the third adhesive section 22c is less than the peel strength of adhesive sections 22a and 22b; thus removal of the removable strip 28 is made easier.

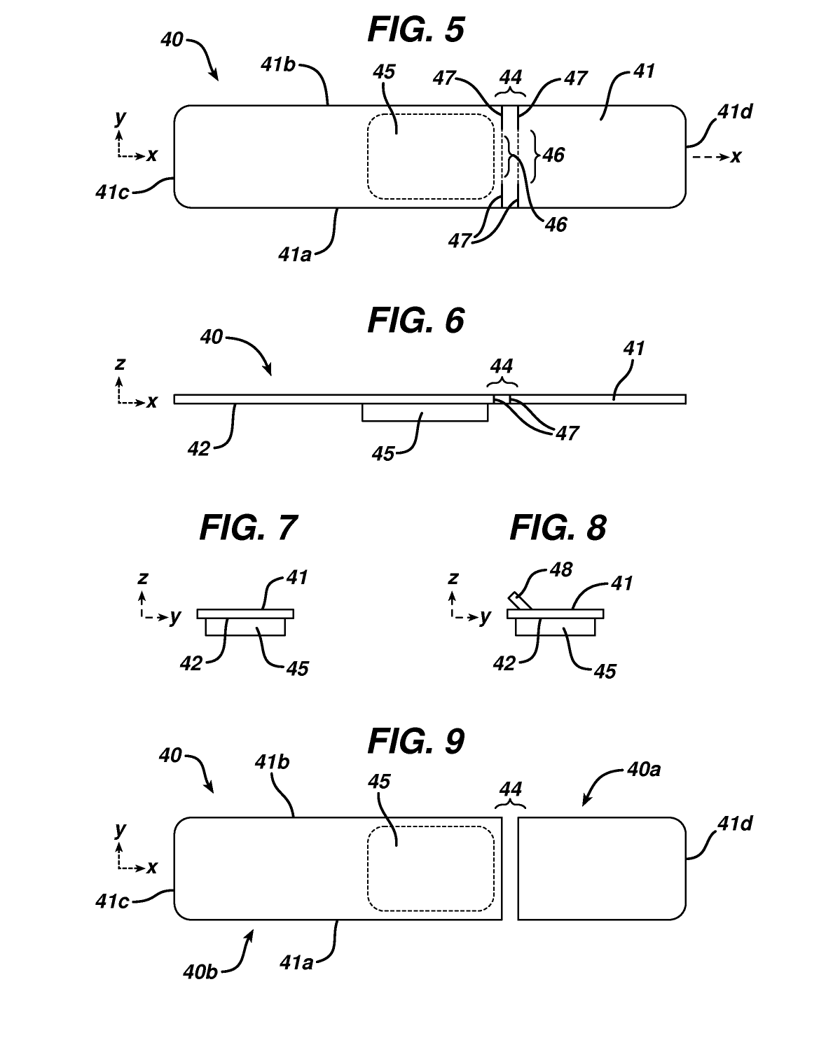

FIGS. 5 through 9 illustrate a third embodiment of an elongate adhesive article of the present invention. In this embodiment, the elongate adhesive article will be referred to as adhesive bandage 40 (also known as a sticking plaster). Bandage 40 comprises backing layer 41 with a first and second surface, an adhesive layer 42 and an absorbent pad 45 associated with the first layer. Backing layer 41 has first side edge 41a, second side edge 41b, first end 41c, and second end 41d. Bandage 40 has a removable strip 49 located proximate the absorbent pad 45 at least partially defined by lines of weakness 46 and two pairs of slits 47 which originate from both first side edge 21a and second side edge 41b, and are disposed along the y-axis. Thus, the removable strip 49 is less likely to be covered by an end of the elongate article when wrapped around a small appendage, such as a finger or toe.

To aid in the removal of bandage 40, user would lift the removable strip 49 starting between the pair of slits 47 using for example, a finger nail. In this manner, the pair of slits 47 acts as a “pick point”, or “starting point”. User would then tear along the lines of weakness 46 starting from one side edge of backing layer 41, across the width of the backing layer, and remove the removable strip 49. The user can start from either first side edge 41a or second side edge 41b of backing layer 41, as each side has a “pick point”. The user can then use the torn edges remaining on bandage 40 as a tab to aid in the removal of the remaining pieces of bandage 40.

Details of backing layer 41, adhesive layer 42, lines of weakness 46, and slits 47 are the same as those discussed in the first and second embodiment (FIGS. 1 through 4). As shown in FIG. 5, lines of weakness 46 and slits 47 are disposed on one side of absorbent pad 45. In other embodiments, lines of weakness 46 and slits 47 may be disposed over the region of the backing layer 41 above absorbent pad 45.

Bandages 40 as shown in the third embodiment have an absorbent pad 45 associated with first surface of the backing layer 41. Absorbent pad 45 includes a first surface facing the second surface of backing layer 41, and that has a first surface area, and a second surface opposite the first surface and facing the skin, and that has a second surface area. As used herein, “associated with the backing layer” means that absorbent pad 45 is affixed either directly or indirectly to backing layer 41 so that it will not become separated from absorbent pad 45 during normal use.

Again, a user can remove the bandage 40 by lifting an edge of the removable strip 49. FIG. 8 is an end view of bandage 40 after the removable strip 49 has been partially lifted (along the z-axis). Partially lifted removable strip 49 is shown as tab 48.

FIG. 9 is a top view of bandage 40 after removable strip 49 has been removed. Bandage 40 is now in two sections, first section 40a and second section 40b. Second section 40b has absorbent pad 45 adhered to it. First section 40a and second section 40b have now been slightly lifted, allowing for easy removal from a substrate.

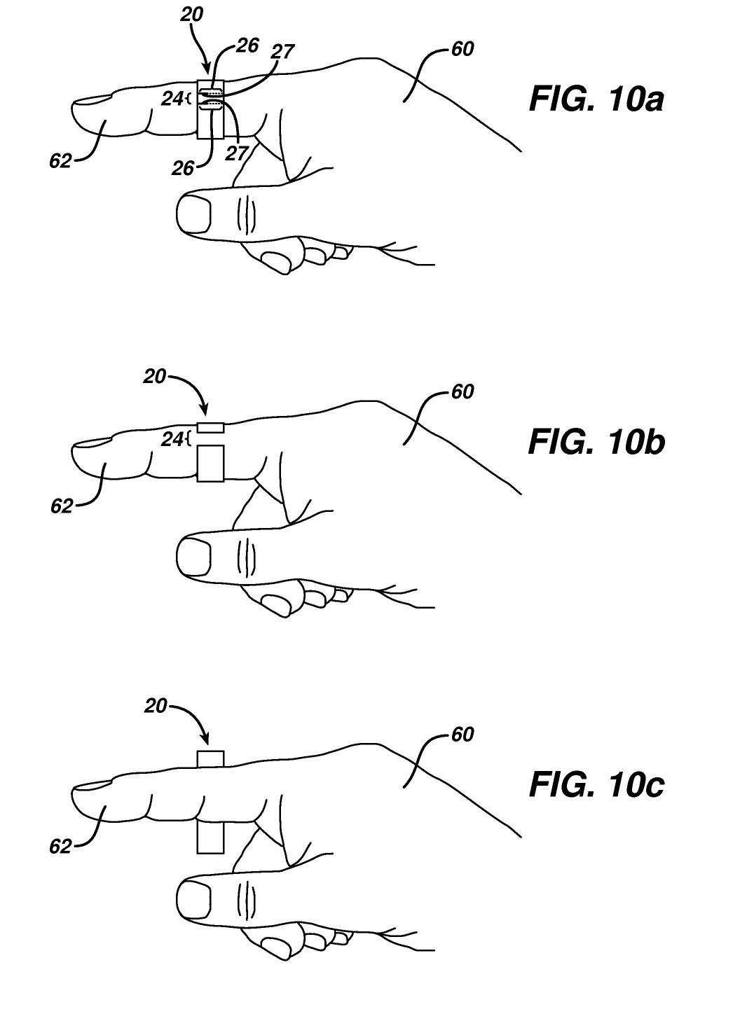

FIGS. 10a to 10c are a representation of one removal method of the second embodiment the elongate adhesive article 20 of the present invention from a user's index finger 62. FIG. 10a shows user's hand 60 and an index finger 62 on which is disposed the elongate adhesive article 20. Removable strip 28 is shown on the elongate adhesive article 20. To remove the elongate adhesive article 20 from finger 62, user would use a finger nail to lift an edge of the removable strip 28 from finger starting between the pair of slits 27 (“pick point”). User would then tear perforations 26 along their length, and remove the removable strip 28. FIG. 10b shows the elongate adhesive article after easy to removable strip 28 has been lifted. The user can then use the torn edges remaining on the elongate adhesive article 20 as a tab to aid in the removal of the remaining pieces of the elongate adhesive article 20 from the index finger 62. FIG. 10c shows the elongate adhesive article 20 partially removed from finger 62.

The process of manufacturing the elongate adhesive articles described above may be any of those conventionally known to produce adhesive bandages. The backing layer and adhesive layer can be obtained by any methods available at present. For example, an extrusion process may be used for obtaining the backing layer. In the same way, the adhesive layer can be made in any known manner. A backing layer as described herein is obtained and an adhesive layer as described herein is applied to the second surface of the backing layer. Optionally, a release layer may be applied to the adhesive layer. The release layer is removed from the adhesive article prior to application by the user.

The process of manufacturing the adhesive bandages articles described above may be similar to the method described above for adhesive articles. The added component, the absorbent pad, can be obtained by any methods available at present. After adhesive layer is applied to the second surface of the backing layer as described herein, the absorbent pad is associated with the adhesive layer, thus bonding the absorbent pad to the backing layer.

The lines of weakness described above may be formed by a variety of manufacturing processes. In some embodiments, mechanical punches may be employed to perforate the materials. Hot pin perforation, also known in the prior art, may be used in the manufacturing process. Kiss cutting and die cutting are also known processes that may be employed to perforate, slit, and/or score.

Ultrasonic perforation is also employed in the prior art. Ultrasonic systems employ ultrasonic equipment adjacent to a pin roll with a fixed gap of space in the path of the web between the ultrasonic equipment and the pin roll. In one embodiment, the ultrasonic system includes a nip roll for providing tension to the web, a pin roll constructed of hardened and/or unhardened steel and a wear resistant coating, and an ultrasonic horn, which is cooled by a stream of forced air. The ultrasonic horn and pin roll are preferably positioned so that there is no gap between the two, and no calibration or extremely precise machining of the pin roll is required. The method of the invention includes holding the web in tension, perforating the web with ultrasonic equipment which is immediately adjacent to a pin roll, and cooling the ultrasonic equipment with a forced stream of air. The resulting material has well defined holes without abnormal tearing, and has a smooth surface with no raised annular edges around the holes.

As mentioned above, in some embodiments the peel strength the weakened region may be less than the peel strength in other regions of the first, adhesive surface. This can be accomplished several ways. An adhesive having a lower peel strength can be applied in the region corresponding to the removable strip by methods such as using a slot coating die. Alternatively, the adhesive layer may be configured in discontinuous patterns on the backing layer, with less, or no adhesive applied in the region corresponding to the removable strip. This can be accomplished using a screen, a spray or any other method which a person skilled in the art understands as to use to form a discontinuous layer of adhesive. In other embodiments, the region of adhesive corresponding to the removable strip can be covered with permanent facings, such as, a thin backing or sheet material of lesser adhesiveness. One such material is plastic netting sold under the trade name DELNET (Delstar Technologies, Middletown, Del.).

The elongate adhesive articles described above may also be ideally suited to deliver one or more active ingredients such as therapeutics to the surface of the skin. When contained in the bandages 40 of the invention, one or more active ingredients may be contained primarily or exclusively in the absorbent pad 45 of bandage 40. Illustrative classes of active ingredients that may be delivered to the skin via the elongate adhesive bandages 40 of the invention include, but are not limited to, antibiotics, analgesics, antipyretics, antimicrobials, antiseptics, antiallergics, anti-acne, anesthetics, anti-inflammatories, hemostats, cosmetics, vitamins, vasodilators, emollients, pH regulators, antipruritics, counterirritants, antihistamines and steroids. Specific active ingredients that may be delivered to the skin via the dressings of the invention include chlorhexidine, neomycin sulfate, polymyxin-B sulfate, zinc bacitracin, benzalkonium chloride, cetylpyridinium chloride, bupivacaine, tetracaine, cincaine, lidocaine, benzocaine, silver sulfadiazine, hydrocortisone, metandienone, trypsin, tolazoline, heparin, pramoxine, aloe vera, tretinoin, retinol, retinaldehyde, menthol, capsaicin, alpha hydroxy acids and vitamins such as Vitamin E.

The present invention will be better understood from a consideration of the following illustrative examples.

### EXAMPLES

### Example 1

Prototypes were handmade using commercially available wound covering bandages. The commercial bandages used are sold under the trademark BAND-AID® Brand Adhesive Bandages by Johnson & Johnson Consumer Products Company (Skillman, N.J.). The bandages used are sold under the names “Sheer Strips” and “Flexible Fabric”. The dimensions of both bandages were 3-inch length by 0.75-inch width.

The process for making prototypes started with removing the outer wrapping of the bandage. Next, the release layer was removed from the adhesive areas. The bandage was placed, adhesive side down, on a smooth polypropylene sheet that was coated with a thin layer of hand cream. The hand cream kept the adhesive side from strongly adhering to the polypropylene sheet. The polypropylene sheet was placed on a stack of about ten 5-cm by 5-cm POST-IT® Note sheets (3M, Minneapolis, Minn.). The sheets served as cushioning for the cutting procedure.

Slits and pick points were created in the bandage backing layer using a model X3602#2 X-ACTO knife (Elmer's Products, Inc., Westerville, Ohio) to cut four slits (two parallel pairs) through the backing layer. The disposition of the slits was similar to the location of slits 47 shown on FIG. 5. Each slit was about 4-mm long, and the distance between parallel slits were about 4-mm. In between the slits the tip of the X-ACTO knife was used to create lines of perforations. The disposition of the perforations was similar to the location of lines of weakness 46 shown on FIG. 5. The perforations were approximately 0.5-1 mm in length, and spaced approximately 0.5-1 mm apart. There were approximately ten perforations per line of perforations.

The process for making prototypes was completed by removing the bandage from the smooth polypropylene sheet, and replacing the release layer on the adhesive area.

To demonstrate use of the prototype bandage, the release layer was removed from the bandage, and the bandage was wrapped, adhesive side down, around the index finger of a user. The bandage was wrapped so that there was an overlap of the backing layer. To remove the bandage, the user used a finger nail from the opposite hand to lift a portion of the releasable strip. The user then tore the perforations along their length, removing the removable strip. The user then used the torn edges of the prototype bandage as a tab to unwrap the rest of the bandage from the index finger.

While various embodiments of the invention have been set forth above, it will be apparent to those skilled in the art that various modifications and variations can be made in the present invention without departing from the scope or spirit of the invention. Thus, it is intended that the present invention cover such modifications and variations as come within the scope of the appended claims and their equivalents.

What is claimed is:

1. An elongate adhesive article having a length substantially greater than a width, the article comprising:
a) a backing layer having disposed on a first surface thereof an adhesive, the backing layer having a thickness, first and second side edges running the length of the article and first and second ends; and
b) a single weakened region comprising a removable strip of backing layer that is oriented substantially perpendicular to the article length; wherein the removable strip of backing layer is at least partially defined by a pair of lines of weakness separated by a gap of between about 2 and about 15 mm.

2. The article of claim 1, wherein the pair of lines of weakness is separated by a gap of between about 2 and about 6 mm.

3. The article of claim 1, wherein the pair of lines of weakness extends from the first side edge toward the second side edge.

4. The article of claim 1, wherein the pair of lines of weakness spans substantially all of the width of the article.

5. The article of claim 1, wherein at least one of the lines of weakness span comprises perforations.

6. The article of claim 1, wherein at least one of the lines of weakness span comprises a reduction in the thickness of the backing layer.

7. The article of claim 1 wherein at least one of the lines of weakness comprises a slit proximate the first side edge.

8. The article of claim 7 wherein the slit is oriented substantially perpendicular to the first side edge.

9. The article of claim 1 wherein the lines of weakness are substantially parallel.

10. The article of claim 1, wherein the first surface of the backing layer has at least two regions, a first region corresponding to the removable strip and a second region corresponding to other portions of the surface, and the first region has a peel strength of the removable strip is less than the peel strength of the second region.

11. The article of claim 1, further comprising an absorbent pad operatively connected to the first surface of the backing layer.

12. The article of claim 11, wherein the absorbent pad comprises one or more materials selected from the group consisting of fibers, foams, and hydrocolloids.

13. The article of claim 12, wherein the absorbent pad comprises a woven or nonwoven fabric.

14. The article of claim 11, wherein the absorbent pad further comprises at least one active ingredient.

15. A method of using the elongate adhesive article of claim 1 comprising the steps of:
a) applying the article to mammalian skin;
b) gripping the removable strip of backing layer proximate the first side edge of the article and pulling the removable strip of backing layer away from the skin to which it is applied, thereby fracturing the backing layer along the lines of weakness and to remove the removable strip of backing layer from the adhesive article and leaving a remainder of the adhesive article adhered to the skin;
c) gripping the remainder of the adhesive article and removing the remainder of the adhesive article from the skin.

16. The method of claim 15, wherein the step of applying the article comprises encircling a mammalian body part with the article and superposing the first end over the second end and adhering the first end to the second end.
